(19) United States
(12) Patent Application Publication
(10) Pub. No.: US 2026/0165793 A1
(43) Pub. Date: Jun. 18, 2026

(54) ICOSAHEDRON MOUNTING GEOMETRY FOR ARRAY MOUNTING TO ARRAY CLAMPS

(71) Applicant: DePuy Ireland Unlimited Company, Ringaskiddy (IE)

(72) Inventors: Ryan Ross, Warsaw, IN (US); Colt Fleming, Warsaw, IN (US); Adam Furore, Warsaw, IN (US); Somasekhar Patnala, Warsaw, IN (US); Paul Rajchel, Warsaw, IN (US); Isaac Stammen, Warsaw, IN (US)

(73) Assignee: DePuy Ireland Unlimited Company, Ringaskiddy (IE)

(21) Appl. No.: 19/407,178

(22) Filed: Dec. 3, 2025

Related U.S. Application Data
(60) Provisional application No. 63/733,132, filed on Dec. 12, 2024.

(51) Int. Cl. A61B 34/20; A61B 90/00

(52) U.S. Cl. CPC A61B 34/20; A61B 90/39; A61B 2034/2072; A61B 2090/3991

(57) ABSTRACT

A system may include a navigation array, a mating interface, and an array clamp. The navigation array may include a frame with a plurality of navigation markers. The mating interface may be configured to mount the navigation array to an array clamp. The mating interface may include a plurality of flat surfaces. The navigational tracking system may include a surgical assistance system that may have a processor configured to track a position of the navigation array to determine a position of the system during a surgical procedure.

### CROSS-REFERENCE TO RELATED APPLICATIONS

This application claims the benefit of U.S. Provisional Patent Application No. 63/733,132, filed on Dec. 12, 2024, the contents of which are incorporated by reference herein in its entirety.

### BACKGROUND

Surgical joint repair procedures involve repair and/or replacement of a damaged or diseased joint. Many times, a surgical joint repair procedure, such as joint arthroplasty as an example, involves replacing the damaged joint with a prosthetic that is implanted into the patient's bone. Proper selection of a prosthetic that is appropriately sized and shaped and proper positioning of that prosthetic to ensure an optimal surgical outcome can be challenging. To assist with positioning, the surgical procedure often involves the use of surgical instruments to control the shaping of the surface of the damaged bone and cutting or drilling of bone to accept the prosthetic.

Virtual visualization tools are available to surgeons that use three-dimensional modeling of bone shapes to facilitate preoperative planning for joint repairs and replacements. These tools can assist surgeons with the design and/or selection of surgical guides and implants that closely match the patient's anatomy and can improve surgical outcomes by customizing a surgical plan for each patient.

Current mechanical mating designs between array clamps and navigation arrays fail to provide a wide degree of freedom while maintaining a solid geometry that would provide rigidity between the array clamp and navigation array when the two are fixed during a surgical procedure.

### SUMMARY

A system may include a navigation array, a mating interface, and an array clamp. The navigation array may include a frame. The frame may include a plurality of navigation markers. The mating interface may be configured to mount the navigation array to an array clamp. The mating interface may include a plurality of flat surfaces. The straight edge may be defined at a boundary between a pair of the plurality of surfaces such that the mating interface defines a plurality of straight edges. Three or more straight edges may meet at a vertex such that the mating interface defines a plurality of vertices. The array clamp may include a mating surface configured to receive the mating interface of the navigation array.

The mating surface of the array clamp may include a clam shell interface that is configured to receive the mating interface of the navigation array.

In one embodiment, the clam shell interface may include two mating pieces connected to each other. The two mating pieces may define a mating cavity configured to receive the mating interface of the navigation array. The mating cavity may define an inner surface. The inner surface of the mating cavity may define a plurality of flat surfaces that have a same shape and a same size as the plurality of flat surfaces of the mating interface of the navigation array.

The mating cavity may be configured to apply opposing forces to opposing flat surfaces of the mating interface of the navigation array to secure the mating interface of the navigation array within the mating cavity. The opposing flat surfaces may be in parallel with one another.

In another embodiment, the system may include a navigation tracking system. The navigational tracking system may include a surgical assistance system. The surgical assistance system may include a processor configured to track a position of the navigation markers to determine a position of the navigation array during a surgical procedure. The navigation tracking system may include any combination of a camera, a robotic system, a virtual reality system, or an augmented reality system.

In one example, a system for navigated surgery may include a navigation array and a mating interface. The navigation array may include a plurality of navigation markers. The mating interface may be configured to mount the navigation array to an array clamp. The mating interface may include a plurality of flat surfaces. A straight edge may be defined at a boundary between a pair of the plurality of surfaces such that the mating interface defines a plurality of straight edges. Three or more straight edges may meet at a vertex such that the mating interface defines a plurality of vertices.

The plurality of flat surfaces may be congruent to each other. Each straight edge of the plurality of straight edges may be the same length, and the plurality of vertices may define equal angles between multiple surfaces of the plurality of flat surfaces. The mating interface may define twenty triangular flat surfaces, thirty edges, and twelve vertices.

The mating interface may define twenty equal-sided triangular flat surfaces arranged to form an icosahedron shape with the plurality of vertices, each of the plurality of vertices being formed by a conversion of sides of five triangular surfaces. The twenty triangular flat surfaces may be arranged such that each triangular surface is spaced from a substantially parallel opposite triangular surface.

The mating interface may be coupled to the navigation array via a neck. The neck may be coupled to a vertex of the plurality of vertices of the mating interface. The navigation array may include three elongated portions. The plurality navigation of markers may be located at junctions between the three elongated portions.

### BRIEF DESCRIPTION OF THE DRAWINGS

FIG. 1A is an example illustrating a navigation array that includes a bullet tip.

FIG. 1B is an illustration of an example array clamp placed on a bone without a navigation array.

FIG. 1C is an illustration of two example array clamps placed on bones with navigation arrays.

FIG. 2 is an example illustrating a navigation array with a mounting geometry.

FIG. 3 is an example perspective view of a mounting geometry of the navigation array.

FIG. 4 is a diagram illustrating an example mounting geometry of the navigation array placed in a mating cavity of the array clamp.

### DETAILED DESCRIPTION

Navigation or tracking of instruments during surgical procedures has become increasingly popular. Surgical navigation can help surgeons avoid delicate neural or vascular structures when moving instruments within a patient. In knee surgery, for example, a surgical navigation system can be used during bone drilling, implant insertion, e.g., screw insertion, and other steps of the surgery. Use of surgical navigation systems can also reduce the amount of X-ray exposure to which the patient and operating room staff are exposed as procedures that do not utilize surgical navigation systems typically perform more steps using fluoroscopy or other X-ray based imaging.

A typical navigation system includes an array of navigation markers attached to a surgical instrument, an imaging system that captures images of the surgical field, and a controller that detects the navigation markers in the captured images and tracks movement of the navigation markers within the surgical field. The controller associates a reference frame of the imaging system with a reference frame of the patient and, informed by a known geometry of the array and the instrument, determines how the instrument is being moved relative to the patient. Based on that determination, the controller provides navigation feedback to the surgeon. The arrays can have different types or geometries, which can vary based on the navigation system, type of surgery, and/or location within the patient that is being tracked.

The precision of the navigation system strongly depends on the design of the tracked instrument and, in particular, the rigidity of the interface between the navigation array and remainder of the instrument. Welding or integrally forming the navigation array to the instrument can result in relatively high precision being achieved. Such solutions, however, can be inconvenient, as the capability to decouple the array from the instrument or to couple the array to other instruments is absent. Further, arrangements having the navigation array integrally formed with the instrument can require separate instruments for standard and navigation use, thereby raising costs for equipment.

A number of solutions have been developed to allow the navigation array to be interchangeably attached with one or more instruments. Such interchangeable connections can have a significant influence on precision of the instrument navigation. Interchangeable connections can include interfaces that have bullet, dovetail or v-groove geometries to connect the navigation array to the instrument. Due to manufacturing tolerances and other variations that prevent perfect mating between the many contacting surfaces in such overdetermined configurations, it can be difficult to consistently and repeatably attach the array and the instrument in a desired relative position and orientation. Current mechanical mating designs that allow the navigation array to be interchangeably attached with one or more instruments lack degree of freedom in relative position and orientation before fastening. When fixed throughout a surgical procedure, the current mechanical mating designs are unable to maintain a solid rigid geometry. Additionally, a bumped array would cause a surgeon to abandon robotic procedure and perform manual surgery.

Accordingly, there is a need for improved devices, systems, and methods to couple a first object and a second object in a repeatable manner that provides a degree of freedom before these two objects are secured, and also securely couple these two objects in a precise manner that maintains a solid rigid geometry after these two objects are secured.

Instrument mating interfaces and related methods are disclosed herein, e.g., for coupling or mounting a navigation array to an array clamp or other component. An embodiment of a coupling of the present disclosure may include an array clamp associated with a first coupling component, such as a mating interface of the array clamp, or a clam shell interface, and a navigation array associated with a second coupling interface, such as a mating interface of the navigation array, a polyhedron, a platonic solid, or an icosahedron. The first coupling component may be configured to mate with the second coupling component such that the second coupling component is disposed in the first coupling component. The second coupling component may have a degree of freedom in relative position and orientation before the first coupling component is fastened. Upon fixation when the second coupling component is fastened onto the first coupling component, the navigation array and the array clamp may be able to securely coupled in a precise manner that maintain a solid rigid geometry. The degree of freedom of this mating interface design eliminates the necessity of a second mating interface within the array clamp, and thus minimize or eliminate navigational inaccuracy associated with system tolerances of the objects and/or components in a navigated instrument system.

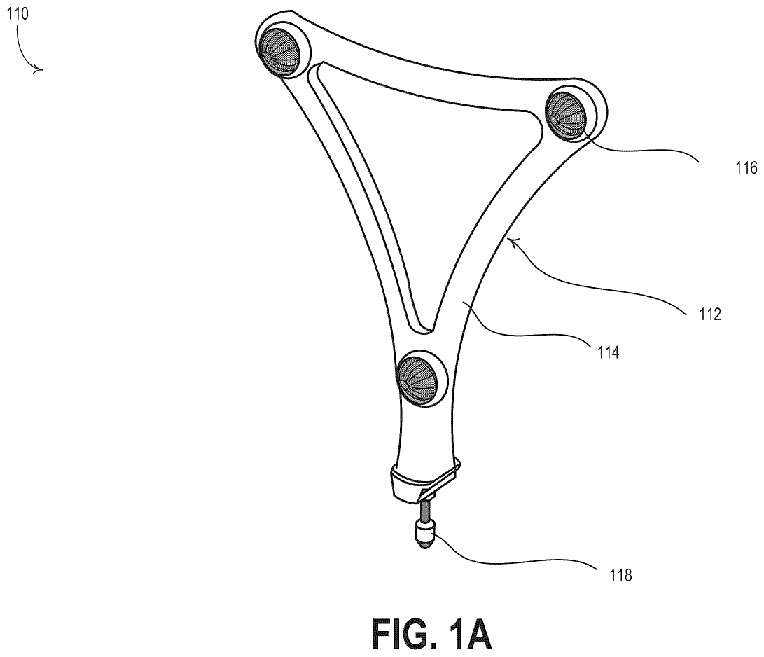

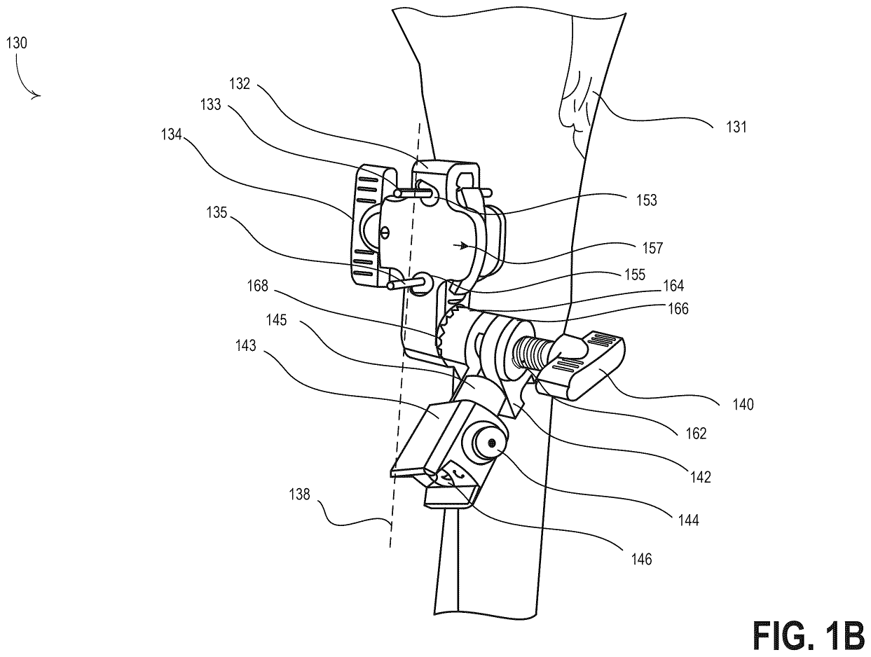

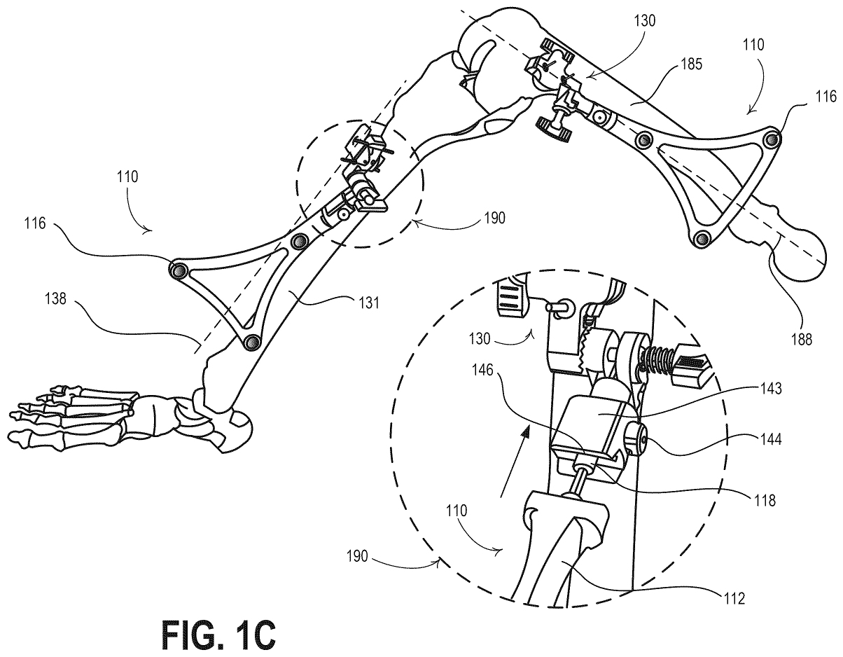

FIGS. 1A to 1C illustrate a navigation array 110 that is configured to be coupled to an array clamp 130 and shown on bone in fixation 150. FIG. 1A illustrates an example a navigation array 110 that includes a mating interface 118, a frame 112, and/or one or more navigation markers 116. The mating interface 118 may be bullet tip shape. The mating interface 118 may be a cylindrical portion that tapers at a distal end of the navigation array 110. The frame 112 or the navigation array 110 may include three elongated portions 114 that extend between two navigation markers 116. The elongated portions 114 may be made of plastic. The elongated portions 114 may be bent with a degree or curvature. One or more of the elongated portions 114 may bow inward such that the elongated portions 114 are concave in shape. In one embodiment, one of the elongated portions 114 may be convex shape. The three elongated portions 114 may form a triangle shape. The formed triangle shape may define three corners. A navigation marker 116 or a reflective element may be located at each corner of the frame 112, where the elongated portions 114 converge. The navigation markers 116 may be reflective spheres.

The navigation markers 116 of the navigation array 110 may be fixed reference points used to create an accurate frame of reference for a system that is configured to assist a navigated surgical procedure. A camera of the system may track the orientation of the navigation array 110 by tracking the navigation markers 116 throughout a surgical procedure. In this way, the system for navigated surgery may “see” a bone or a knee, for example, the system may determine a relative location of the bone or knee, by monitoring the position or movement of the navigation markers 116.

FIG. 1B is an illustration of the array clamp 130 placed on a bone 131 without a navigation array, such as the navigation array 110. The array clamp 130 may include an array clamp body 132, an array clasp 142, and an array clamp tip 143. The array clamp body 132 may include a first array drill pin 133, a pin wingnut 134, a second array drill pin 135, a first array drill pin hole 153, a second array drill pin hole 155, and an arrow 157.

A surgeon may first perform a first stab incision into skin at an intended location of the first array drill pin 133. The first array drill pin 133 may then be drilled perpendicular and through the center of the bone 131 to ensure the first array drill pin 133 is rigidly attached to the bone 131. The array clamp body 132 may then be placed over the first array drill pin 133, using the first array drill pin hole 153. The arrow 157 on the array clamp body 132 may be pointing towards the camera (not shown). The pin wingnut 134 may be pointing away from the camera (not shown). The surgeon may then perform a second stab incision at the intended location of the second array drill pin 135. The surgeon may use the second array drill pin hole 155 as a guide to drill the second array drill pin 135 parallel to the first array drill pin 133 to avoid stress on the bone 131 when adjusting a position of the array clamp body 132. The second array drill pin 135 may also be rigidly attached to the bone 131. The first array drill pin 133 and the second array drill pin 135 may be aligned with a tibial long axis 138. The tibial long axis 138 may be an axis along the bone 131 (e.g., that is parallel to the bone 131). The pin wingnut 134 may be rotated to tighten the array clamp body 132 onto the first array drill pin 133 and the second array drill pin 135. A navigation array, for example, the navigation array 110 as shown in FIG. 1A, may be attached to an array clamp 130 that is rigidly fixed to array drill pins 133 and 135. The array drill pins 133 and 135 may be fixed to a femur or tibia at a beginning of the navigated surgery.

The array clamp tip 143 may include a button 144, array clamp tip head 145, and an array clamp tip hole 146. The array clamp tip head 145 may be inserted into an array clasp 142. In one embodiment, when the array clamp tip head 145 is inserted into the array clasp 142, the button 144 on the array clamp tip 143 may be pointing away from the bone 131. After a surgeon secured the array clamp tip 143 onto the array clasp 142, the surgeon may press the button 144 and insert a mating interface of a navigation array, for example, the bullet tip shape mating interface 118 of the navigation array 110 as shown in FIG. 1A, into the array clamp tip hole 146. Upon release of the button 144, the mating interface of the navigation array may be rigidly fixed onto the array clamp tip 143.

The array clasp 142 may include an array wingnut 140, a wingnut screw 162, a first clamshell mating part 164 and a second clamshell mating part 166. The array wingnut 140 may be connected to the wingnut screw 162. The array clasp 142 may be secured onto the array clamp body 132 via the wingnut screw 162. A surgeon may rotate the array wingnut 140, causing the wingnut screw 162 connected to the array wingnut 140 to go through the clamshell mating parts 166 and 164 into the array clamp body 132. The first clamshell mating part 164 of the array clasp 142 may be in contact with the array clamp body 132 at an array clamp joint 168. The array clamp tip head 145 of the array clamp tip 143 may be between the clamshell mating parts 164 and 166 within the array clasp 142. A surgeon may further rotate or tighten the array wingnut 140 so that the clamshell mating parts 164 and 166 may squeeze the array clamp tip head 145, causing the array clamp tip 143 to be rigidly attached to the array clasp 142, and the array clasp 142 is rigidly fixed to the array clamp body 132.

The assembly of the array clamp 130 may have three connections. A navigation array, for example the navigation array 110 as shown in FIG. 1, may be connected to the array clamp tip 143. The array clamp tip 143 may be connected to the array clasp 142. The array clasp 142 may be connected to the array clamp body 132. For the connection between the navigation array to the array clamp tip 143, after a mating interface of the navigation array, for example the mating interface 118 of the navigation array 110, is inserted into the array clamp tip hole 146 of the array clamp tip 143, the mating interface may be rigidly fixated onto a cavity defined by the array clamp tip 143 and may have no freedom of movement. For the connection between the array clamp tip 143 and the array clasp 142, after the array clamp tip head 145 is inserted into the array clasp 142 and before the array clamp tip head 145 is rigidly fixed onto the array clasp 142, the array clamp tip head 145 may have a degree of freedom to rotate left and right within the cavity formed by the clamshell mating parts 164 and 166. For the connection between the array clasp 142 and the array clamp body 132, after the array clasp 142 is attached to the array clamp body 132 via the wingnut screw 162 and before the array wingnut 140 is further tightened, the array clasp 142 may have a degree of freedom to move up and down about the array clamp joint 168.

These three connections may offer the navigation array the degree of freedom to move up and down, as well as the degree of freedom to rotate left and right in respect to the array clamp body 132. However, due to manufacturing tolerances and other variations that prevent perfect mating between the many contacting surfaces in such overdetermined configurations, it can be difficult to consistently and repeatably attach the array and the instrument in a desired relative position and orientation. Current mechanical mating designs for array fixation may lack degree of freedom while maintaining a solid geometry because the solid geometry may provide rigidity when fixed throughout a surgery. Any movement during the surgery may result in inaccuracy in actual cuts compared to cuts in a plan. Additionally, a bumped array would cause a surgeon to abandon robotic procedure and perform manual surgery. Thus, there may be a need to reduce the number of connections so that manufacturing tolerances and other variations that may prevent perfect mating may be reduced.

FIG. 1C is an illustration of two array clamps 130 each with a navigation array 110 coupled to the array clamp 130. The array clamps 130 are fixed to a tibia 131 and to a femur 185, respectively. Section 190 is a perspective view of the coupling interface between the array clamp 130 and the navigation array 110 illustrating the coupling of the navigation array 110 to the array clamp 130. The array clamp 130 may include an array clamp tip 143 that is configured to receive the mating interface 118 of the navigation array 110. The array clamp tip 143 may define the array clamp tip hole 146 that is sized to receive the mating interface 118. The array clamp 130 may include the button 144 that is configured to be actuated to release the mating interface 118 of the navigation array 110.

The navigation array 110 may include the mating interface 118, for example, the bullet tip shape mating interface 118 as shown in FIG. 1A. The navigation array 110 may include the frame 112. The surgeon may press the button 144 and insert the mating interface 118 of the navigation array 110 through the array clamp tip hole 146 and into a cavity defined by the array clamp tip 143. Upon release of the button 144, the mating interface 118 of the navigation array 110 may be rigidly fixed onto the array clamp tip 143.

The array clamp 130 may be fixed to the tibia 131 and/or fixed to the femur 185. As described in FIG. 1B, the two array drill pins may be aligned with a tibial long axis 138 when the array clamp 130 is fixed to the tibia 131. The navigation array 110 may have three navigation markers 116. When the array clamp 130 is fixed to the femur 185, the two array drill pins may be aligned with a femoral long axis 188.

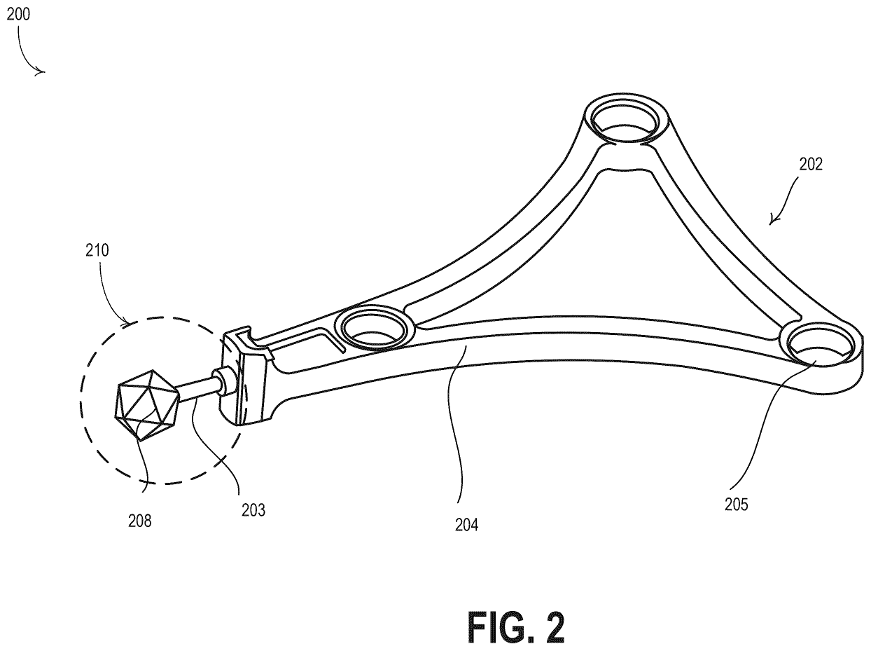

FIGS. 2 to 4 illustrates an example navigation array 200 and mating surface 220 of an array clamp. The navigation array 200 and the mating surface 220 may provide a degree of freedom while maintaining a solid geometry. The solid geometry may provide rigidity when the solid geometry is fixed throughout a surgery.

The navigation array 200 may include a frame 202, one or more navigation marker cavities 205, and a mounting geometry 210. The frame 202 may include three elongated portions 204. The elongated portions 204 may be made of plastic. The elongated portions 204 may be bent such that they define a convex or concave curvature. For example, one or more of the elongated portions 204 may bow inward such that the elongated portions 204 are concave in shape. In one embodiment, one of the elements may be convex shape (not shown). The elongated portions 204 may form a triangle shape that defines three corners. A navigation marker cavity 205 may be located at each corner of the frame 202, where the elongated portions 204 converge. Each navigation marker cavity 205 may be configured to receive a navigation marker (e.g., such as the navigation marker 116 of FIG. 1A). The navigation marker may be a reflective sphere.

The navigation markers may be fixed reference points used to create an accurate frame of reference for a system that is configured to assist a navigated surgical procedure. The system may include the navigation array 200, an array clamp (e.g., such as the array clamp 130 and/or an array clamp that includes the mating surface 220), and a navigational tracking system (e.g., a surgical assistance system). The navigational tracking system may track a position of the navigation array to determine a position of the system during a surgical procedure. The navigation tracking system may include any combination of one or more processors, a camera, a robotic system, a virtual reality system, or an augmented reality system. The one or more processors may use feedback from the camera to track the orientation of the navigation array 200 by tracking the navigation markers throughout a surgical procedure. In this way, the system for navigated surgery may “see” a bone or a knee, for example, the system may identify a relative location of the bone or knee, by monitoring the position or movement of the navigation markers. Examples of navigational tracking systems that include one or more processors, a camera, a robotic system, a virtual reality system, an augmented reality system, and related tracking units are described in U.S. Patent Application publication no. US 2021/0100629 A1, which is incorporated by reference herein in its entirety. The navigation arrays described herein (e.g., the navigation array 200) may be used with (e.g., as active markers or trackers in) the navigational tracking systems described in U.S. Patent Application publication no. US 2021/0100629 A1.

The mounting geometry 210 may include a mating interface 208 and a neck 203. The mating interface 208 may be located at a distal end of the navigation array 200, and may be coupled to the frame 202 or the navigation array 200 via a neck 203. The neck 203 may be connected to the frame 202 on one end and the mating interface 208 on another end. The mating interface 208 may have various geometric shapes.

In one example, the mating interface 208 may be a polyhedron (e.g., may define a polyhedron). The polyhedron may have a plurality of flat surfaces, and the plurality of flat surfaces may have boundaries with straight edges. The mating interface 208 may be formed by closed surfaces with polygonal faces. When the polyhedron shaped mating interface is inserted to an array clasp (e.g., such as an array clasp that includes the mating surface 220), the mating interface may be securely fixated onto the array clamp.

The mating interface 208 may be a convex regular polyhedron, or a platonic solid. Similar to the polyhedron, the platonic solid may have a plurality of flat surfaces, boundaries, and straight edges. The platonic solid may be symmetrical. The platonic solid shaped mating interface may have a plurality of vertices. The flat surfaces of the platonic solid may have equal size and shape. The boundaries of the flat surfaces and the straight edges may be of equal lengths. The plurality of vertices may be the same. When the platonic solid shaped mating interface is inserted to an array clasp and before an array wingnut (e.g., such as the array wingnut 140 of FIG. 1B) is tightened, the mating interface may be able to rotate up, down, left and right, and thus provide the navigation array 200 a degree of freedom to move about the array clasp to allow a surgeon to position or orient the navigation array 200 relative to the patient prior to surgery. The navigation array 200 may have different fixation positions when the navigation array 200 is fastened onto the array clamp, for example, by leveraging the clamping force that can be applied by the mating surface of the array clasp to opposing surfaces of the polyhedron.

In one embodiment, the mating interface 208 may be a polyhedron with twenty triangular flat surfaces, thirty edges, and twelve vertices. The mating interface 208 may be a regular icosahedron. When the icosahedron shaped mating interface is inserted to an array clasp and before an array wingnut is tightened, the mating interface may the navigation array 200 a degree of freedom to move about the array clasp. After an array wingnut is tightened, the mating interface may also be rigidly secured onto an array clamp. In some examples, the mating interface 208 may be a polyhedron with between 12-28 equally sized flat surfaces. In other examples, the mating interface 208 may be a polyhedron with between 16-24 equally sized flat surfaces. In the illustrated example, the mating interface 208 may be an icosahedron with 20 equally sized flat surfaces.

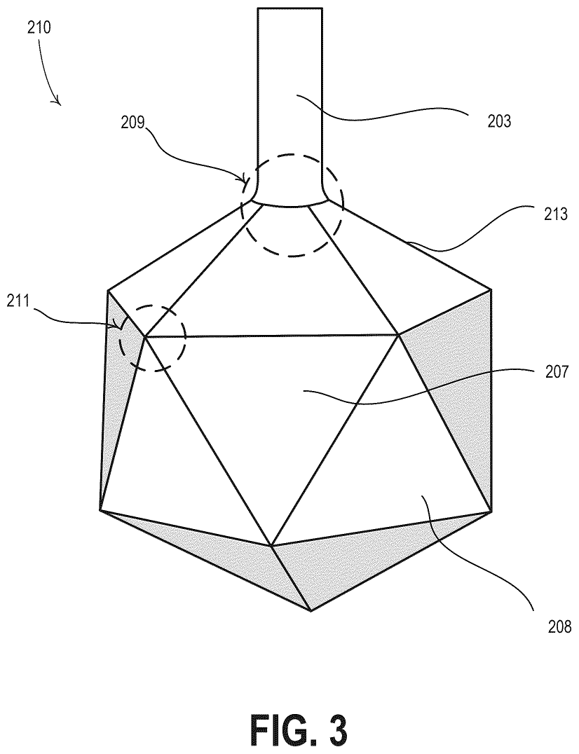

FIG. 3 is an example perspective view of the mounting geometry 210 of the navigation array 200. The shading in FIG. 3 is used to show depth. The mounting geometry 210 may include the neck 203 and the mating interface 208. The mating interface 208, as shown in FIG. 2, may include a plurality of flat surfaces 207. The flat surfaces 207 may have boundaries. In one embodiment, the boundaries may be straight edges 213. A plurality of the straight edges 213 may meet at a plurality of vertices 211. The straight edge 213 may be at a boundary between a pair of the plurality of flat surfaces 207 such that the mating interface 208 defines a plurality of straight edges 213. The three or more straight edges 213 may meet at a vertex such that the mating interface 208 defines a plurality of vertices 211.

The plurality of flat surfaces 207 may be congruent to each other. Each straight edge of the plurality of straight edges 213 may be the same length. The plurality of vertices 211 may define equal angles between multiple surfaces of the plurality of flat surfaces 207. In one embodiment, the mating interface 208 may define twenty triangular flat surfaces, thirty edges, and/or twelve vertices.

The mating interface 208 may define twenty equal-sided triangular surfaces 207 arranged to form an icosahedron shape with the plurality of vertices 211. Each of the plurality of vertices 211 may be formed by a conversion of sides of five triangular surfaces 207. The twenty triangular flat surfaces 207 may be arranged such that each triangular surface is spaced from a substantially parallel opposite triangular surface.

The mating interface 208 may be coupled to a frame (not shown), for example, the frame 202 of the navigation array 200 as shown in FIG. 2, via the neck 203. The neck 203 may be coupled to a vertex 209 of the plurality of vertices of the mating interface 208.

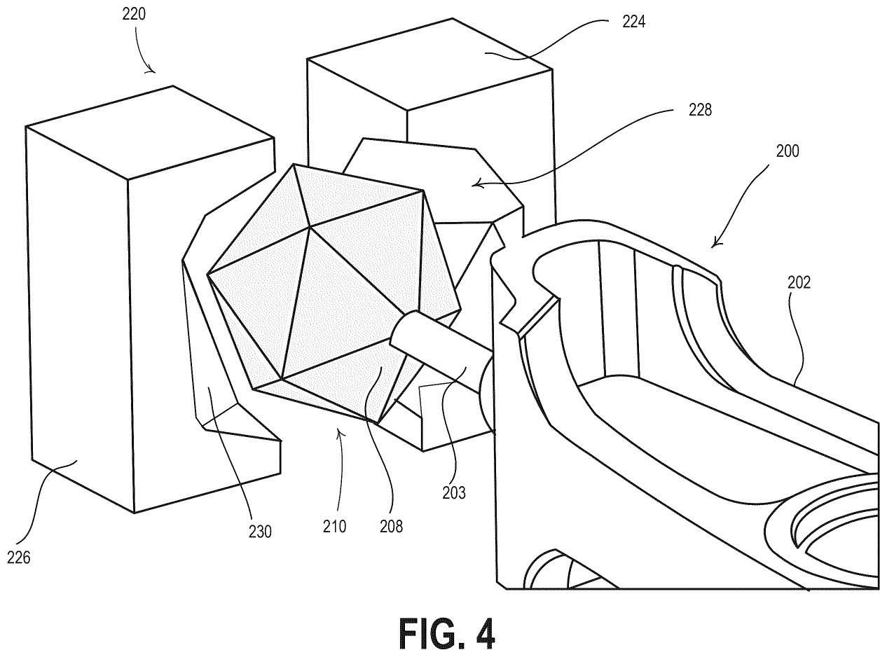

FIG. 4 is an illustration of the example mounting geometry 210 of the navigation array 200 and a mating cavity 228 of a mating surface 220 of an array clamp. An example of the array clamp (not shown) that includes a mating surface 220 may be the array clamp 130 as shown in FIG. 1B. The mating cavity 228 may be configured to receive the mating interface 208 of the navigation array 200. The mating surface 220 may include two mating pieces 224, 226 that are hinged or coupled together (not shown). The inner surfaces of two mating pieces 224, 226 may define the mating cavity 228 that is configured to receive the mating interface 208 of the navigation array 200. The mating surface 220 of the array clamp may be a clam shell interface that is configured to receive the mating interface 208 of the navigation array 200. For example, although not illustrated, the two mating pieces 224, 226 may be configured such that the distance between the two mating pieces 224, 226 can be widened and narrowed such that the mating surface 220 can securely couple to the mating interface 208 of the navigation array 200.

The mating cavity 228 may define an inner surface 230. The inner surface 230 of the mating cavity 228 may define a plurality of flat surfaces that have the same shape and size as the plurality of flat surfaces of the mating interface 208 of the navigation array 200. The mating cavity 228 may be configured to apply opposing forces to opposing flat surfaces of the mating interface 208 of the navigation array 200 to secure the mating interface 208 of the navigation array 200 within the mating cavity 228 in a plurality of different positions or orientations.

As such, the navigation array 200 that includes a polyhedron shaped mating interface may allow for an increased number or degree of freedom of movement of the orientation of the navigation array 200 relative to the array clamp. This allows a large degrees of motion or freemen of positioning of the navigation array 200 by the physician relative to the array clamp and surgical site prior to starting the surgical procedure. As such, the navigation array 200 can be optimally position for tracking while also being position such that it does not interfere with the surgical procedure. This is, in part, enabled through the use of a polyhedron shaped mating interface 208. The polyhedron (e.g., the icosahedron having 20 faces) allows the navigation array 200 to be placed in multiple geometries where it can be viewed by the camera, but also present a solid surface where once tightened down so that the navigation array 200 is securely coupled to the array clamp (e.g., to prevent any slight movements or deviation from that original fixation during surgery).

For instance, after the mating interface 208 is inserted into the array clasp and before the mating interface 208 is rigidly fixed onto the array clasp, the mating interface 208 may have a degree of freedom to rotate left, right, up and down within the cavity formed by the clamshell mating parts 224 and 226. After the array clasp is attached to the array clamp body (e.g., via an wingnut screw, such as the wingnut screw 162 of FIG. 1B) and before an array wingnut (e.g., such as the array wingnut 140 of FIG. 1B) is further tightened, the array clasp may have a degree of freedom to move up and down about an array clamp joint (e.g., such as the array clamp joint 168 of FIG. 1B). These two connections with mating interface 208 in the current disclosure may reduce manufacturing tolerances and other variations, in comparison to the three connections used in prior art. These two connections may preserve or increase degree of freedom of the navigation array 200. These two connections may offer the navigation array 200 the degree of freedom to move up and down, as well as the degree of freedom to rotate left and right in respect to the array clamp body. The two connections with mating interface 208 may provide rigidity when fixed throughout a surgery, and may also increase accuracy during a surgical procedure.

1. A system comprising:
an array clamp; and
a navigation array comprising a frame and a mating interface;
wherein the frame comprises a plurality of navigation markers;
wherein the mating interface is configured to mount the navigation array to the array clamp, wherein the mating interface comprises a plurality of flat surfaces, wherein a straight edge is defined at a boundary between a pair of the plurality of flat surfaces such that the mating interface defines a plurality of straight edges, and wherein three or more straight edges meet at a vertex such that the mating interface defines a plurality of vertices; and
wherein the array clamp comprises a mating surface configured to receive the mating interface of the navigation array.

2. The system of claim 1, wherein the plurality of flat surfaces are congruent to each other, wherein each straight edge of the plurality of straight edges are the same length, and wherein the plurality of vertices define equal angles between multiple surfaces of the plurality of flat surfaces.

3. The system of claim 2, wherein the mating interface defines twenty triangular flat surfaces, thirty edges, and twelve vertices.

4. The system of claim 1, wherein the mating interface defines twenty equal-sided triangular flat surfaces arranged to form an icosahedron shape with the plurality of vertices, each of the plurality of vertices being formed by a conversion of sides of five triangular surfaces, wherein the twenty equal-sided triangular flat surfaces are arranged such that each triangular surface is spaced from a substantially parallel opposite triangular surface.

5. The system of claim 1, wherein the mating interface is coupled to the navigation array via a neck.

6. The system of claim 5, wherein the neck is coupled to a vertex of the plurality of vertices of the mating interface.

7. The system of claim 1, wherein the navigation array comprises three elongated portions, wherein the plurality of navigation markers are located at junctions between the three elongated portions.

8. The system of claim 1, wherein the mating surface of the array clamp comprises a clam shell interface that is configured to receive the mating interface of the navigation array.

9. The system of claim 8, wherein the clam shell interface comprises two mating pieces connected to each other, the two mating pieces defining a mating cavity configured to receive the mating interface of the navigation array.

10. The system of claim 9, wherein the mating cavity defines an inner surface, wherein the inner surface of the mating cavity defines a plurality of flat surfaces that have a same shape and a same size as the plurality of flat surfaces of the mating interface of the navigation array.

11. The system of claim 9, wherein the mating cavity is configured to apply opposing forces to opposing flat surfaces of the mating interface of the navigation array to secure the mating interface of the navigation array within the mating cavity.

12. The system of claim 11, wherein the opposing flat surfaces are in parallel with one another.

13. The system of claim 1, further comprising:
a navigational tracking system comprising:
a surgical assistance system comprising a processor configured to track a position of the navigation markers to determine a position of the navigation array during a surgical procedure.

14. The system of claim 13, wherein the navigation tracking system comprises any combination of a camera, a robotic system, a virtual reality system, or an augmented reality system.

15. A system for navigated surgery, comprising:
a navigation array comprising a plurality of navigation markers; and
a mating interface configured to mount the navigation array to an array clamp, wherein the mating interface comprises a plurality of flat surfaces, wherein a straight edge is defined at a boundary between a pair of the plurality of flat surfaces such that the mating interface defines a plurality of straight edges, and wherein three or more straight edges meet at a vertex such that the mating interface defines a plurality of vertices.

16. The system for navigated surgery of claim 15, wherein the plurality of flat surfaces are congruent to each other, wherein each straight edge of the plurality of straight edges are the same length, and wherein the plurality of vertices define equal angles between multiple surfaces of the plurality of flat surfaces.

17. The system for navigated surgery of claim 16, wherein the mating interface defines twenty triangular flat surfaces, thirty edges, and twelve vertices.

18. The system for navigated surgery of claim 15, wherein the mating interface defines twenty equal-sided triangular flat surfaces arranged to form an icosahedron shape with the plurality of vertices, each of the plurality of vertices being formed by a conversion of sides of five triangular surfaces, wherein the twenty equal-sided triangular flat surfaces are arranged such that each triangular surface is spaced from a substantially parallel opposite triangular surface.

19. The system for navigated surgery of claim 15, wherein the mating interface is coupled to the navigation array via a neck, and wherein the neck is coupled to a vertex of the plurality of vertices of the mating interface.

20. The system for navigated surgery of claim 15, wherein the navigation array comprises three elongated portions, wherein the plurality of navigation markers are located at junctions between the three elongated portions.
